(19) United States
(12) Patent Application Publication
(10) Pub. No.: US 2023/0405023 A1
(43) Pub. Date: Dec. 21, 2023

(54) USE OF THE NAV1.6 SODIUM CHANNEL BLOCKER (S)-4-((1-BENZYLPYRROLIDIN-3-YL)(METHYL)AMINO)-2-FLUORO-5-METHYL-N(THIAZOL-4-YL)BENZENESULFONAMIDE, TOGETHER WITH STRONG INDUCERS OF CYTOCHROME P450 3A4, IN THE TREATMENT OF CONDITIONS ASSOCIATED WITH NAVI.6 ACTIVITY

(71) Applicant: Xenon Pharmaceuticals Inc., Burnaby (CA)

(72) Inventors: Gregory N. Beatch, West Vancouver (CA); Rostam Namdari, Burnaby (CA)

(21) Appl. No.: 18/248,663

(22) PCT Filed: Apr. 9, 2021
(86) PCT No.: PCT/CA2021/050472

§ 371 Date: Apr. 11, 2023

Related U.S. Application Data
(60) Provisional application No. 63/090,866, filed on Oct. 13, 2020.

(51) Int. Cl. A61K 31/635; A61K 31/4166

(52) U.S. Cl. CPC A61K 31/635; A61K 31/4166

(57) ABSTRACT

Provided are methods for treating diseases or conditions associated with Nav1.6 activity in a patient, comprising administering to said patient (S)-4((1-benzylpyrrolidin-3-yl)(methypamino)-2-fluoro-5-methyl-N(thiazol-4-yl)benzenesulfonamide or a pharmaceutically acceptable salt thereof, wherein the patient is also being administered a strong inducer of cytochrome P450 3A4 (CYP3A4).

This application claims priority to, and the benefit of, U.S. Application No. 63/090,866, which was filed on Oct. 13, 2021, the entirety of which is incorporated by reference herein.

Voltage gated sodium channels (Nay's) are critical determinants of cellular excitability in muscle and nerve. Four isoforms in particular, Nav1.1, Nav1.2, Nav1.3, and Nav1.6, account for the majority of sodium current in the neurons of the central nervous system. Nav1.3 is primarily expressed embryonically. Beyond the neonatal stage, Nav1.1, Nav1.2, and Nav1.6 are the critical isoforms that regulate neuronal signaling in the brain.

Epilepsy is a condition characterized by excessive synchronous excitability in the brain that arises when the delicate balance of excitatory and inhibitory signals in the brain fall out of equilibrium. This can happen either due to an excess of excitation, or a deficiency of inhibition. Mutations in the genes encoding Nav channels have been linked to both types of disequilibrium.

Phenytoin may be the most commonly used antiepileptic medication in North America. However, phenytoin is known to be an inducer of certain cytochrome P450 (CYP450) enzymes. The CYP450 enzyme system is responsible for the biotransformation of drugs from active substances to inactive metabolites that can be excreted from the body. In addition, the metabolism of certain drugs by CYP450 can alter their pharmacokinetic profile and result in sub-therapeutic plasma levels of those drugs over time.

Despite the advances that have been made in this field, there remains a need for new therapeutic products useful to treatment of epilepsy and other diseases or conditions associated with Nav1.6 activity and particularly, therapeutic products that can be co-administered with phenytoin or other inducers of the cytochrome P450 3A4 (CYP3A4) enzyme. The present disclosure fulfills these and other needs, as evident in reference to the following disclosure.

### SUMMARY

Provided is a method of treating a disease or a condition associated with Nav1.6 activity in a patient wherein the patient is also being administered a strong inducer of cytochrome P450 3A4 (CYP3A4), the method comprising:
administering to the patient in need thereof a therapeutically effective amount of (S)-4-((l-benzylpyrrolidin-3-yl)(methyl)amino)-2-fluoro-5-methyl-N-(thiazol-4-yl)benzenesulfonamide (Compound A), or a pharmaceutically acceptable salt thereof.

Also provided is a method of treating epilepsy in a patient wherein the patient is also being administered a therapeutically effective amount of phenytoin, the method comprising:
administering to the patient in need thereof a therapeutically effective amount of (S)-4-((l-benzylpyrrolidin-3-yl)(methyl)amino)-2-fluoro-5-methyl-N-(thiazol-4-yl)benzenesulfonamide (Compound A), or a pharmaceutically acceptable salt thereof.

Also provided is a method of treating a disease or a condition associated with Nav1.6 activity in a patient, the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
subsequently determining that the patient is to begin treatment with a strong inducer of cytochrome P450 3A4 (CYP3A4), and
continuing administration of Compound A, or a pharmaceutically acceptable salt thereof, to the patient.

Also provided is a method of treating a disease or a condition associated with Nav1.6 activity in a patient wherein the patient is also being administered a strong inducer of cytochrome P450 3A4 (CYP3A4), the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
wherein the administration produces a median time to peak plasma concentrations (T max) for Compound A, or a pharmaceutically acceptable salt thereof, that is about the same for a patient who is not being administered a strong inducer of CYP3A4 than the median Tₘₐₓ for Compound A, or a pharmaceutically acceptable salt thereof, for a patient who is not being administered a strong inducer of CYP3A4.

Also provided is a method of treating a disease or a condition associated with Nav1.6 activity in a patient wherein the patient is also being administered a strong inducer of cytochrome P450 3A4 (CYP3A4), the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
wherein the administration an area under the curve from time 0 to the last sampling time point (AUC₀₋ₜ) and to infinity (AUC_{0-inf}) for Compound A, or a pharmaceutically acceptable salt thereof, that is about the same for a patient who is not being administered a strong inducer of CYP3A4, than the administration an AUC₀₋ₜ and to AUC_{0-inf} for Compound A, or a pharmaceutically acceptable salt thereof, for a patient who is not being administered a strong inducer of CYP3A4.

These and other objects of the invention are described in the following paragraphs. These objects should not be deemed to narrow the scope of the invention.

### DETAILED DESCRIPTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### A. Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used in the specification and the appended claims, unless specified to the contrary, the following terms have the meaning indicated:

As used in the specification and the appended claims, unless specified to the contrary, the following terms have the meaning indicated:

The term “API” as used herein stands for “active pharmaceutical ingredient.” The API as disclosed herein is (S)-4-((1-benzylpyrrolidin-3-yl)(methyl)amino)-2-fluoro-5-methyl-N-(thiazol-4-yl)benzenesulfonamide (Compound A) or a pharmaceutically acceptable salt thereof. Compound A may also be referred to as XEN901 or NBI 921352.

The term “inducer” of enzyme activity when the specific activity or the metabolic effect of the specific activity of the enzyme can be increased by the presence of the substance, without reference to the precise mechanism of such increase. For example, a substance can be an inducer of enzyme activity by increasing reaction rate, by increasing expression of the enzyme, by allosteric activation or other direct or indirect mechanisms. Co-administration of a given drug with an enzyme inducer may increase the rate of excretion of the drug metabolized through the pathway indicated.

As used herein, a “strong CYP3A4 inducer” is a compound that decreases the area under the concentration time curve (AUC) of a sensitive index substrate of the CYP3A4 pathway by ≥80%. Index substrates predictably exhibit exposure increase due to inhibition or induction of a given metabolic pathway and are commonly used in prospective clinical drug-drug interaction studies. Sensitive index substrates are index substrates that demonstrate an increase in AUC of ≥5-fold with strong index inhibitors of a given metabolic pathway in clinical drug-drug interaction studies. Examples of sensitive index substrates for the CYP3A pathway are midazolam and triazolam. See, e.g., Drug Development and Drug Interactions: Table of Substrates, Inhibitor and Inducers at https://www.fda.gov/drugs/developmentapprovalprocess/developmentresources/druginteractionslabeling/ucm093664.htm.

Lists of inhibitors, inducers and substrates for CYP3A4 can be found, for instance, at http://www.genemedrx.com/Cytochrome P450 Metabolism Table.php, and other sites and http://www.ildcare.eu/downloads/artseninfo/drugs metabolized by cyp450s.pdf.

As used herein, the term “pharmaceutical composition” means a composition comprising Compound A or a pharmaceutically acceptable salt thereof and, optionally, one or more pharmaceutically acceptable excipients.

The term “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree.

The term “patient” or “individual” or “subject” means a mammal, including a human, for whom or which therapy is desired, and generally refers to the recipient of the therapy.

The term “effective amount” and “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

The terms “treating” or “treatment” refers to therapeutic applications to slow or stop progression of a disorder, prophylactic application to prevent development of a disorder, and/or reversal of a disorder. Reversal of a disorder differs from a therapeutic application which slows or stops a disorder in that with a method of reversing, not only is progression of a disorder completely stopped, cellular behavior is moved to some degree, toward a normal state that would be observed in the absence of the disorder.

The term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The term “administering to a patient” refers to the process of introducing a composition or dosage form into the patient via an art-recognized means of introduction.

The term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The term “administering to a patient” refers to the process of introducing a composition or dosage form into the patient via an art-recognized means of introduction.

The term “t_{1/2}” or “plasma half-life” or “elimination half-life” or the like is a pharmacokinetic parameter denoting the apparent plasma terminal phase half-life, i.e., the time, after absorption and distribution of a drug is complete, for the plasma concentration to fall by half.

The term “AUC” refers to the area under the curve, or the integral, of the plasma concentration of an active pharmaceutical ingredient or metabolite over time following a dosing event.

The term “AUC₀₋ₜ” is the integral under the plasma concentration curve from time 0 (dosing) to time “t”.

The term “AUC_{0-∞}” is the AUC from time 0 (dosing) to time infinity. Unless otherwise stated, AUC refers to AUC_{0-∞}.

The term “adjusting administration”, “altering administration”, “adjusting dosing”, or “altering dosing” are all equivalent and mean tapering off, reducing or increasing the dose of the substance, ceasing to administer the substance to the patient, or substituting a different active agent for the substance.

The term “informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

The term “labeling” means all labels or other means of written, printed, graphic, electronic, verbal, or demonstrative communication that is upon a pharmaceutical product or a dosage form or accompanying such pharmaceutical product or dosage form.

The term “a medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

The term “Medication Guide” means an FDA-approved patient labeling for a pharmaceutical product conforming to the specifications set forth in 21 CFR 208 and other applicable regulations which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

The term “patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

The term “product” or “pharmaceutical product” means a dosage form of an active agent plus published material, and optionally packaging.

The term “product insert” means the professional labeling (prescribing information) for a pharmaceutical product, a patient package insert for the pharmaceutical product, or a medication guide for the pharmaceutical product.

The term “professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

The term “published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

The term “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

The term “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

The term “immediate release” pharmaceutical formulation includes any formulation in which the rate of release of drug from the formulation and/or the absorption of drug, is neither appreciably, nor intentionally, retarded by galenic manipulations. Thus, the term excludes formulations which are adapted to provide for “modified”, “controlled”, “sustained”, “prolonged”, “extended” or “delayed” release of drug. In this context, the term “release” includes the provision (or presentation) of drug from the formulation to the gastrointestinal tract, to body tissues and/or into systemic circulation.

The term “stable” refers to both chemical (shelf-life) and physical stability (suspension uniformity). Improved uniformity results in an improved product because less shaking of the suspension is required before dosing and allows the product to be stored longer (i.e. longer shelf-life) because the drug in the product will not settle and compact.

The term “co-administer” and “co-administration” and variants thereof refers to the administration of at least two drugs to a patient either subsequently, simultaneously, or consequently proximate in time to one another (e.g., within the same day, or week or period of 30 days, or sufficiently proximate that each of the at least two drugs can be simultaneously detected in the blood plasma). When co-administered, two or more active agents can be co-formulated as part of the same composition or administered as separate formulations. This also may be referred to herein as “concomitant” administration or variants thereof.

### B. Drug Substance

Pharmaceutical compositions disclosed herein comprise at least one active pharmaceutical ingredient: (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof.

Compound A has the following formula:

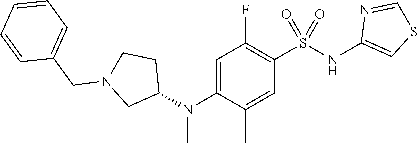 [SMILES: Cc1cc(S(=O)(=O)Nc2cscn2)c(F)cc1N(C)[C@H]1CCN(Cc2ccccc2)C1]

Methods of making Compound A and a pharmaceutically acceptable salt thereof are described in U.S. Pat. No. 10,662,184, the contents of which are herein incorporated by reference.

Compound A has been reported to be metabolized by CYP3A4, CYP2D6, and to a lesser extent CYP2C9.

Compound A may be present in a pharmaceutical composition in the form of acid addition salts. Acid addition salts of the free amino compounds may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, p-toluenesulfonic acid, and benzenesulfonic acids. Suitable inorganic acids include hydrochloric, hydrobromic, sulfuric, phosphoric, and nitric acids. Thus, the term “pharmaceutically acceptable salt” of Compound A is intended to encompass any and all acceptable salt forms.

As used herein, and in the absence of a specific reference to a particular pharmaceutically acceptable salt of Compound A, any dosages, whether expressed in milligrams or as a percentage by weight or as a ratio with another ingredient, should be taken as referring to the amount of Compound A. For example, a reference to “20 mg Compound A or a pharmaceutically acceptable salt thereof” means an amount of Compound A or a pharmaceutically acceptable salt thereof that provides the same amount of Compound A as 20 mg of Compound A free form.

In some embodiments, Compound A, or a pharmaceutically acceptable salt thereof, is Compound A free base.

In some embodiments, Compound A, or a pharmaceutically acceptable salt thereof, is a pharmaceutically acceptable salt of Compound A.

In some embodiments, the amount of Compound A, or pharmaceutically acceptable salt thereof, is from about 2 mg to about 100 mg. In some embodiments, the amount of Compound A is about 2.5, about 5, about 10, about 20, about 30, about 40, or about 50 mg. In some embodiments, the amount of Compound A is about 2.5, about 5, about 10, or about 20 mg. In some embodiments, the amount of Compound A is about 2.5 mg. In some embodiments, the amount of Compound A is about 5 mg. In some embodiments, the amount of Compound A is about 10 mg. In some embodiments, the amount of Compound A is about 20 mg. In some embodiments, the amount of Compound A is about 30 mg. In some embodiments, the amount of Compound A is about 40 mg. In some embodiments, the amount of Compound A is about 50 mg. In some embodiments, the amount of Compound A is about 60 mg. In some embodiments, the amount of Compound A is about 70 mg. In some embodiments, the amount of Compound A is about 80 mg. In some embodiments, the amount of Compound A is about 90 mg. In some embodiments, the amount of Compound A is about 100 mg.

In some embodiments, Compound A, or the pharmaceutically acceptable salt thereof, is present in an amount of between about 5 and about 15% w/w, measured as the free base. In some embodiments, Compound A, or the pharmaceutically acceptable salt thereof, is present in an amount of about 10% w/w, measured as the free base.

### C. The Co-Administered Drug

In some embodiments, Compound A, or the pharmaceutically acceptable salt thereof, is co-administered with a strong inducer of CYP3A4.

In some embodiments, the strong inducer of CYP3A4 is chosen from apalutamide, carbamazepine, dexamethasone, enzalutamide, fosphenytoin, lumacaftor, midostaurin, mitotane, pentobarbital, phenobarbital, phenytoin, primidone, rifampicin, rifamycin, rifaximin, rimexolone, and St. John's Wort.

The co-administered drug is administered in accordance with prescribing information for such agents as set forth, for example, in a package insert. For example, the prescribing information for DILANTIN® (phenytoin sodium) indicates that it is indicated for the treatment of tonic-clonic (grand mal) and psychomotor (temporal lobe) seizures and prevention and treatment of seizures occurring during or following neurosurgery and includes the following dosage and administration information:
Adult starting dose in patients who have received no previous treatment is one 100 mg DILANTIN extended capsule three times a day, with dose adjustments as necessary. For most adults, the satisfactory maintenance dose will be one capsule three to four times a day. An increase, up to two capsules three times a day may be made, if necessary.
Adult once-a-day dose: If seizure control is established with divided doses of three 100 mg DILANTIN extended capsules daily, once-a-day dosage with 300 mg DILANTIN extended capsules may be considered.
Pediatric starting dose is 5 mg/kg/day in two to three equally divided doses, with dosage adjustments as necessary, up to a maximum of 300 mg daily. Maintenance dosage is 4 to 8 mg/kg/day.
Serum blood level determinations may be necessary for optimal dosage adjustments—the clinically effective serum total concentration is 10-20 mcg/mL (unbound phenytoin concentration is 1-2 mcg/mL).

In some embodiments, the co-administered drug is phenytoin. In some embodiments, 100 mg phenytoin, measured as the free base, is administered 3 times a day (TID). In some embodiments, an extended release formulation of 300 mg phenytoin, measured as the free base, is administered once-a-day. In some embodiments, when the patient is a pediatric patient, the starting dose of phenytoin is 5 mg/kg/day in two to three equally divided doses, with dosage adjustments as necessary, up to a maximum of 300 mg daily. Maintenance dosage is 4 to 8 mg/kg/day. In some embodiments, the phenytoin is administered in a manner to deliver a serum total concentration of 10-20 mcg/mL (unbound phenytoin concentration is 1-2 mcg/mL).

### C. Pharmaceutical Compositions

Administration of Compound A, or a pharmaceutically acceptable salt thereof, and of the co-administered drug can be carried out via any of the accepted modes of administration of agents for serving similar utilities. Administration of the two compounds may be via the same route or different routes.

The pharmaceutical compositions can be prepared by combining a compound with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols.

Typical routes of administering such pharmaceutical compositions include, without limitation, oral, topical, transdermal, inhalation, parenteral, sublingual, rectal, vaginal, and intranasal. The term “parenteral” as used herein includes subcutaneous injections, intravenous, intramuscular, intrasternal injection or infusion techniques. Pharmaceutical compositions are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound for treatment of a disease or condition of.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in Remington's Pharmaceutical Sciences (Mack Pub. Co., N.J. current edition).

A pharmaceutical composition may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a compound such that a suitable dosage will be obtained.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition in solid or liquid form may include an agent that binds to the compound and thereby assists in the delivery of the compound. Suitable agents that may act in this capacity include a monoclonal or polyclonal antibody, a protein or a liposome.

The pharmaceutical composition may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions may be prepared by methodology well known in the pharmaceutical art. For example, a pharmaceutical composition intended to be administered by injection can be prepared by combining a compound with sterile, distilled water so as to form a solution. A surfactant may be added to facilitate the formation of a homogeneous solution or suspension. Surfactants are compounds that non-covalently interact with the compound so as to facilitate dissolution or homogeneous suspension of the compound in the aqueous delivery system.

In some embodiments, the composition of Compound A, or a pharmaceutically acceptable salt thereof, is a granular formulation suitable for reconstitution with a pharmaceutically acceptable carrier to form a stable suspension oral dosage form comprising one or more pharmaceutically acceptable excipients and an amount of (S)-4-((1-benzylpyrrolidin-3-yl)(methyl)amino)-2-fluoro-5-methyl-N-(thiazol-4-yl)benzenesulfonamide (Compound A) or a pharmaceutically acceptable salt thereof.

In some embodiments, the composition of Compound A, or a pharmaceutically acceptable salt thereof, is granular formulation suitable for reconstitution with a pharmaceutically acceptable carrier to form a stable suspension oral dosage form comprising:

(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
at least one pharmaceutically acceptable filler or diluent,
at least one pharmaceutically acceptable binder, and
at least one pharmaceutically acceptable lubricant.

In some embodiments, the composition of Compound A, or a pharmaceutically acceptable salt thereof, is granular formulation suitable for reconstitution with a pharmaceutically acceptable carrier to form a stable suspension oral dosage form comprising:

(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of between about 5 and 15% w/w;
at least one pharmaceutically acceptable filler or diluent, in an amount of between about 55 and about 75% w/w;
at least one pharmaceutically acceptable binder, in an amount of between about 15 and about 30% w/w; and
at least one pharmaceutically acceptable lubricant, in an amount of between about 0.5 and about 2% w/w.

In some embodiments, the composition of Compound A, or a pharmaceutically acceptable salt thereof, is a granular formulation suitable for reconstitution with a pharmaceutically acceptable carrier to form a stable suspension oral dosage form comprising:

(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of about 10% w/w;
at least one pharmaceutically acceptable filler or diluent, in an amount of between about 60 and about 70% w/w;
at least one pharmaceutically acceptable binder, in an amount of between about 20 and about 30% w/w; and
at least one pharmaceutically acceptable lubricant, in an amount of between between about 0.5 and about 1.5% w/w.

In some embodiments, the composition of Compound A, or a pharmaceutically acceptable salt thereof, is a granular formulation suitable for reconstitution with a pharmaceutically acceptable carrier to form a stable suspension oral dosage form comprising:

(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of about 10% w/w;
at least one pharmaceutically acceptable filler or diluent, in an amount of about 64% w/w;
at least one pharmaceutically acceptable binder, in an amount of about 25% w/w; and
at least one pharmaceutically acceptable lubricant, in an amount of between between about 1% w/w.

In some embodiments, the granular formulation suitable for reconstitution comprises:

| | |
|---|---|
| | Strength (label claim) |
| Component and Quality | 50 mg |

| Standard (and Grade, if | Quantity per unit | |
|---|---|---|
| applicable) | (mg) | % |
| Compound A (GMP Grade) | 50.00 | 10.00 |
| Mannitol, Pearlitol 100SD (USP, | 318.75 | 63.75 |
| Ph. Eur., JP) | | |
| Pregelatinized Starch, Starch 1500 | 125.00 | 25.00 |
| (USP-NF, Ph. Eur., JP) | | |
| Grape Flavor | 1.25 | 0.25 |
| Sodium Stearyl Fumarate (USP- | 3.75 | 0.75 |
| NF, Ph. Eur., JP) - Intragranular | | |
| Sodium Stearyl Fumarate (USP- | 1.25 | 0.25 |
| NF, Ph. Eur., JP) - Extragranular | | |

In some embodiments, the composition of Compound A, or a pharmaceutically acceptable salt thereof, is a stable suspension oral dosage form comprising the granular formulation as described herein that has been reconstituted with a pharmaceutically acceptable carrier.

In some embodiments, the composition of Compound A, or a pharmaceutically acceptable salt thereof, is a multiparticulate sprinkle dosage form comprising a plurality of discrete units, wherein each unit comprises one or more pharmaceutically acceptable carriers and an amount of (S)-4-((1-benzylpyrrolidin-3-yl)(methyl)amino)-2-fluoro-5-methyl-N-(thiazol-4-yl)benzenesulfonamide (Compound A) or a pharmaceutically acceptable salt thereof.

In some embodiments, the composition of Compound A, or a pharmaceutically acceptable salt thereof, is a multiparticulate sprinkle dosage form comprising a plurality of discrete units, wherein each unit comprises:

(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
at least one pharmaceutically acceptable filler,
at least one pharmaceutically acceptable binder, and
at least one pharmaceutically acceptable lubricant.

In some embodiments, the composition of Compound A, or a pharmaceutically acceptable salt thereof, is a multiparticulate sprinkle dosage form comprising:

(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of between about 5 and about 15% w/w;
at least one pharmaceutically acceptable filler, in an amount of between about 55 and about 75% w/w;
at least one pharmaceutically acceptable binder, in an amount of between 15 and about 30% w/w; and
at least one pharmaceutically acceptable lubricant, in an amount of between between about 0.5 and about 2% w/w.

In some embodiments, the composition of Compound A, or a pharmaceutically acceptable salt thereof, is a multiparticulate sprinkle dosage form comprising:

(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of about 10% w/w;
at least one pharmaceutically acceptable filler, in an amount of between about 60 and about 70% w/w;
at least one pharmaceutically acceptable binder, in an amount of between about 20 and about 30% w/w; and
at least one pharmaceutically acceptable lubricant, in an amount of between between about 0.5 and about 1.5% w/w.

In some embodiments, the composition of Compound A, or a pharmaceutically acceptable salt thereof, is a multiparticulate sprinkle dosage form comprising:

(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of about 10% w/w;
at least one pharmaceutically acceptable filler, in an amount of about 64% w/w;
at least one pharmaceutically acceptable binder, in an amount of about 25% w/w; and
at least one pharmaceutically acceptable lubricant, in an amount of between between about 1% w/w.

In some embodiments, the composition of Compound A, or a pharmaceutically acceptable salt thereof, is a multiparticulate sprinkle dosage form comprises:

| | Amount |
|---|---|
| Ingredients | (% w/w) |
| Active Ingredient | |
| Compound A | 10.00 |
| Inactive Ingredients | |
| Mannitol | 63.75 |
| Pregelatinized Starch | 25.00 |
| Flavoring Agent | 0.25 |
| Sodium Stearyl Fumarate | 1.00 |
| Total Theoretical Weight | 100.00 |
| Hard gelatin sprinkle capsule, Size 0° | 1 Capsule |

### D Methods of Use

Provided is a method of treating a disease or a condition associated with Nav1.6 activity in a patient wherein the patient is also being administered a strong inducer of cytochrome P450 3A4 (CYP3A4), the method comprising:

administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof.

Also provided is a method of treating epilepsy in a patient wherein the patient is also being administered a therapeutically effective amount of phenytoin, the method comprising:
administering to the patient in need thereof a therapeutically effective amount of (S)-4-((l-benzylpyrrolidin-3-yl)(methyl)amino)-2-fluoro-5-methyl-N-(thiazol-4-yl)benzenesulfonamide (Compound A), or a pharmaceutically acceptable salt thereof.

Also provided is a method of treating a disease or a condition associated with Nav1.6 activity in a patient, the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
subsequently determining that the patient is to begin treatment with a strong inducer of cytochrome P450 3A4 (CYP3A4), and
continuing administration of Compound A, or a pharmaceutically acceptable salt thereof, to the patient.

Also provided is a method of treating a disease or a condition associated with Nav1.6 activity in a patient wherein the patient is also being administered a strong inducer of cytochrome P450 3A4 (CYP3A4), the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
wherein the administration produces a median time to peak plasma concentrations (T max) for Compound A, or a pharmaceutically acceptable salt thereof, that is about the same for a patient who is not being administered a strong inducer of CYP3A4 than the median Tₘₐₓ for Compound A, or a pharmaceutically acceptable salt thereof, for a patient who is not being administered a strong inducer of CYP3A4.

In some embodiments, the median Tₘₐₓ was about 1 hour.

Also provided is a method of treating a disease or a condition associated with Nav1.6 activity in a patient wherein the patient is also being administered a strong inducer of cytochrome P450 3A4 (CYP3A4), the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
wherein the administration an area under the curve from time 0 to the last sampling time point (AUC₀₋ₜ) and to infinity (AUC_{0-inf}) for Compound A, or a pharmaceutically acceptable salt thereof, that is about the same for a patient who is not being administered a strong inducer of CYP3A4, than the administration an AUC₀₋ₜ and to AUC_{0-inf} for Compound A, or a pharmaceutically acceptable salt thereof, for a patient who is not being administered a strong inducer of CYP3A4.

In some embodiments, the method further comprises informing the patient or a medical care worker that co-administration of Compound A, or a pharmaceutically acceptable salt thereof, with a strong inducer of CYP3A4 results in no significant changes in the pharmacokinetics of Compound A.

In some embodiments, the method further comprising informing the patient or a medical care worker that co-administration of Compound A, or a pharmaceutically acceptable salt thereof, with a strong inducer of CYP3A4 results in no significant changes in the pharmacodynamics of Compound A.

In some embodiments, the method further comprises informing the patient or a medical care worker that dose adjustment is not necessary when Compound A, or a pharmaceutically acceptable salt thereof, is co-administered with a strong inducer of CYP3A4.

In some embodiments, the disease or a condition associated with Nav1.6 activity is epilepsy.

In some embodiments, the disease or a condition is selected from photosensitive epilepsy, self-induced syncope, intractable epilepsy, Angelman syndrome, benign rolandic epilepsy, CDKL5 disorder, childhood and juvenile absence epilepsy, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures+, Rett syndrome, multiple sclerosis, Alzheimer's disease, autism, ataxia, hypotonia and paroxysmal dyskinesia.

In some embodiments, the disease or condition is epilepsy with focal onset seizures.

In some embodiments, the disease or condition is epilepsy with generalized onset seizures.

In some embodiments, the disease or condition is epilepsy with unknown onset seizures.

In some embodiments, the disease or condition is epileptic syndrome associated with mutations in SCN8A.

In some embodiments, the disease or condition is Dravet syndrome.

In some embodiments, Compound A, or a pharmaceutically acceptable salt thereof, is administered orally.

In some embodiments, the patient is an adult patient.

In some embodiments, the patient is an adult and is administered between about 75 and 200 mg Compound A, or a pharmaceutically acceptable salt thereof, daily.

In some embodiments, the patient is an adult and is administered between about 25 and 100 mg Compound A, or a pharmaceutically acceptable salt thereof, three times daily.

In some embodiments, the patient is a pediatric patient.

In some embodiments, the patient is a pediatric patient of at least 2 years of age and is administered Compound A, or a pharmaceutically acceptable salt thereof, as shown in the table below.

| | Body | | | |
|---|---|---|---|---|
| Weight | Weight | Dose Level | Dose Level | Dose Level |
| Group | (kg) | 1 (mg) TID | 2 (mg) TID | 3 (mg) TID |
| 1 | 10 to 20 | about 5 | about 10 | about 17.5 |
| 2 | >20 to 35 | about 10 | about 20 | about 30 |
| 3 | >35 to 50 | about 15 | about 30 | about 40 |
| 4 | >50 to 90 | about 20 | about 40 | about 60 |

TID = three times a day

In some embodiments, the patient is a pediatric patient of at least 2 years of age and is administered Compound A, or a pharmaceutically acceptable salt thereof, as shown in the table below.

| | Body | | | |
|---|---|---|---|---|
| Weight | Weight | Dose Level | Dose Level | Dose Level |
| Group | (kg) | 1 (mg) BID | 2 (mg) BID | 3 (mg) BID |
| 1 | 10 to 20 | about 7.5 | about 15 | about 25 |
| 2 | >20 to 35 | about 15 | about 30 | about 45 |
| 3 | >35 to 50 | about 22.5 | about 45 | about 60 |
| 4 | >50 to 90 | about 30 | about 60 | about 90 |

BID = twice a day

In some embodiments, the patient is an adult patient and is administered Compound A or a pharmaceutically acceptable salt thereof in an amount of about 25 mg TID (75 mg/day), about 30 mg TID (90 mg/day), about 35 mg TID (105 mg/day), about 40 mg TID (120 mg/day), about 45 mg TID (135 mg/day), about 50 mg TID (150 mg/day), about 55 mg TID (165 mg/day), about 60 mg TID (180 mg/day), about 65 mg TID (195 mg/day), about 70 mg TID (210 mg/day), about 75 mg TID (225 mg/day), about 80 mg TID (240 mg/day), about 85 mg TID (255 mg/day), about 90 mg TID (270 mg/day), about 95 mg TID (285 mg/day), or about 100 mg TID (300 mg/day).

In some embodiments, the patient is an adult patient and is administered Compound A or a pharmaceutically acceptable salt thereof in an amount of about 30 mg BID (60 mg/day), about 35 mg BID (70 mg/day), about 40 mg BID (80 mg/day), about 45 mg BID (90 mg/day), about 50 mg BID (100 mg/day), about 55 mg BID (110 mg/day), about 60 mg BID (120 mg/day), about 65 mg BID (130 mg/day), about 70 mg BID (140 mg/day), about 75 mg BID (150 mg/day), about 80 mg BID (160 mg/day), about 85 mg BID (170 mg/day), about 90 mg BID (180 mg/day), about 95 mg BID (190 mg/day), about 100 mg BID (200 mg/day), about 110 mg BID (220 mg/day), about 120 mg BID (240 mg/day), about 130 mg BID (260 mg/day), about 140 mg BID (280 mg/day), or about 150 mg BID (300 mg/day).

### F. Examples

1. Pharmacokinetic Study

In this open-label, randomized study, subjects received the following treatments:
Two single oral doses of 100 mg (2×50 mg) Compound A adult immediate release tablet formulation, fasted state, administered on Day 1 and Day 12.
Repeat doses of phenytoin 100 mg TID for 10 days (from Day 3 until the morning of Day 12).

Subjects fasted overnight for a minimum of 10 hours prior to dosing on Day 1 and Day 12. An oral dose of the Compound A IR tablet treatment was administered in the morning with approximately 240 mL of water at ambient temperature, to 1 subject per 2 minute intervals. Fasting continued for at least 4 hours following administration. Water was provided as needed until 1 hour pre-dose. Water was allowed beginning 1 hour after the administration of the drug.

On Day 3 to Day 11, an oral dose of phenytoin was administered TID with approximately 240 mL of water at ambient temperature, starting at 07:30, 15:30, and 23:30 to 1 subject per 2 minute intervals. On Day 12, an oral dose of phenytoin was administered in the morning with approximately 240 mL of water at ambient temperature, starting at 07:30 (1 hour prior to Compound A dosing) to 1 subject per 2 minute intervals.

The direct measurements of this study were the plasma, whole blood, urine, and saliva concentrations of Compound A and plasma concentrations for phenytoin.

Compound A time to peak plasma concentrations (Tₘₐₓ) was approximately 1 h following single-dose administration of Compound A in combination with the administration of phenytoin (a strong CYP3A4 inducer) 100 mg TID for 10 days and following Compound A single-dose administration alone.

Compound A peak plasma concentration (Cₘₐₓ) increased by approximately 22% (geometric mean ratio [GMR] [90% CI]: 121.52% [90.94-162.38%]) when co-administered with phenytoin compared with administration of Compound A alone. The mean Cₘₐₓ values were 3297 ng/mL and 3720 ng/mL, respectively. The intra-subject CV % was 53%.

Compound A total systemic exposure, as indicated by the AUC₀₋ₜ and AUC_{0-inf}, were comparable between treatments with respective mean values of 19139 ng*h/mL and 19816 ng*h/mL for Compound A (100 mg) alone and 17850 ng*h/mL and 18046 ng*h/mL for Compound A (100 mg)+Phenytoin (100 mg TID×10 days). A GMR (with associated 90% CI) of 96.01% (82.54-111.67%) and 92.75% (81.75-105.23%) were observed for AUC₀₋ₜ and AUC_{0-inf}, respectively. The intra-subject CV % were 26% and 22%, respectively.

Compound A elimination half-life values were 10 h and 8 h, apparent volume of distribution values were 89 L and 65 L, and apparent clearance values were 5.4 L/h and 5.7 L/h following administration of Compound A alone or with phenytoin, respectively.

Following the single-dose administration of Compound A in combination with the administration of phenytoin (a strong CYP3A4 inducer) 100 mg TID for 10 days compared to Compound A single-dose administration alone, the mean amount and percentage of the Compound A dose administered that was excreted unchanged in urine over a period of 48 hours following dosing were 71103 ng and 0.07% for Compound A (100 mg) and 57186 ng and 0.06% for Compound A (100 mg)+Phenytoin (100 mg TID).

Compound A CLr was comparable between treatments with mean values of approximately 3-4 mL/h (GMR: 100.25%).

Cₘₐₓ, Tₘₐₓ, and AUC₀₋ₜ parameters were similar when compared from plasma values. Following administration of Compound A (100 mg) alone, Cₘₐₓ were 3216 and 3297 ng/mL and AUC 19942 and 19139 ng*h/mL, for the truncated and the full sampling respectively and Tₘₐₓ was 1 h for both sampling schedule. Following administration of Compound A with Phenytoin (100 mg TID×10 days), Cₘₐₓ values were 3677 and 3720 ng/mL, AUC values were 18590 and 17850 ng*h/mL, for the truncated and the full sampling respectively and Tₘₐₓ values were 1 h for both sampling schedule

The magnitude of DDI was described using GMR (with/without phenytoin) and associated 90% CI of the Cₘₐₓ, AUC₀₋ₜ, and AUC_{0-inf} for Compound A.

Following administration of phenytoin 100 mg TID for 10 days (Day12), the Compound A GMR for AUC₀₋ₜ and AUC_{0-inf} values were similar, differing by only approximately 4% (GMR 96.01%) and 7% (GMR 92.75%) respectively, when compared to Compound A administration alone. The 90% CI of the GMR for Compound A AUC₀₋ₜ and AUC_{0-inf} were within the no effect boundaries (80.00-125.00%). These results showed that phenytoin 100 mg TID did not affect Compound A total systemic exposure. In addition, the mean Cₘₐₓ values increased for Compound A by approximately 22% (GMR 121.52%) when compared to Compound A administration alone. The upper limit of the 90% CI of the GMR for Compound A Cₘₐₓ was above the no effect boundary of 125.00% (90% CI upper limit: 162.38%). Median time to reach Cₘₐₓ (Tₘₐₓ) (1 h) and CLr (3-4 mL/h) remained unchanged in the presence (Day 12) or absence (Day 1) of phenytoin (100 mg TID for 10 days).

Following oral administration of phenytoin 100 mg TID (clinical index strong inducer of CYP3A4) in healthy adult subjects, no dose adjustment of Compound A is required when co-administered with phenytoin or other strong inducers of CYP3A4.

A total of 15 subjects (83%) reported 101 TEAEs over the course of the study. Three treatment periods were defined: Day 1 to Day 3 (following Compound A dosing on Day 1 and prior to start of phenytoin dosing on Day 3) corresponds to the Compound A treatment period, Day 3 to Day 12 (following phenytoin dosing on Day 3 and prior to Compound A dosing on Day 12) corresponds to the phenytoin treatment period, and Day 12 to the follow-up visit (following Compound A dosing on Day 12) corresponds to the Compound A in combination with phenytoin treatment period.

The incidence of subjects with TEAEs was 39% following the administration of Compound A alone on Day 1, 71% following the administration of phenytoin alone from Day 3 to Day 12, and 65% following the administration of Compound A in combination with phenytoin on Day 12. The majority of TEAEs experienced by subjects were considered related to the study drug (Compound A or phenytoin) (95/101; 94%) and were deemed mild (88/101; 87%) in intensity. None of the subjects reported a severe TEAE. No deaths or SAEs occurred in the study, and no subject was withdrawn by the Investigator due to a TEAE (safety reasons).

The pharmaceutical compositions, methods, and uses described herein will be better understood by reference to the following exemplary embodiments and examples, which are included as an illustration of and not a limitation upon the scope of the invention.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

1. A method of treating a disease or a condition associated with Nav1.6 activity in a patient wherein the patient is also being administered a strong inducer of cytochrome P450 3A4 (CYP3A4), the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof.

2. A method of treating epilepsy in a patient wherein the patient is also being administered a therapeutically effective amount of phenytoin, the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof.

3. A method of treating a disease or a condition associated with Nav1.6 activity in a patient, the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
subsequently determining that the patient is to begin treatment with a strong inducer of cytochrome P450 3A4 (CYP3A4), and
continuing administration of Compound A, or a pharmaceutically acceptable salt thereof, to the patient.

4. A method of treating a disease or a condition associated with Nav1.6 activity in a patient wherein the patient is also being administered a strong inducer of cytochrome P450 3A4 (CYP3A4), the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
wherein the administration produces a median time to peak plasma concentrations (Tₘₐₓ) for Compound A, or a pharmaceutically acceptable salt thereof, that is about the same for a patient who is not being administered a strong inducer of CYP3A4 than the median Tₘₐₓ for Compound A, or a pharmaceutically acceptable salt thereof, for a patient who is not being administered a strong inducer of CYP3A4.

5. The method of claim 4, wherein the median Tₘₐₓ was about 1 hour.

6. A method of treating a disease or a condition associated with Nav1.6 activity in a patient wherein the patient is also being administered a strong inducer of cytochrome P450 3A4 (CYP3A4), the method comprising:
administering to the patient in need thereof a therapeutically effective amount of (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
wherein the administration an area under the curve from time 0 to the last sampling time point (AUC₀₋ₜ) and to infinity (AUC_{0-inf}) for Compound A, or a pharmaceutically acceptable salt thereof, that is about the same for a patient who is not being administered a strong inducer of CYP3A4, than the administration an AUC₀₋ₜ and to AUC_{0-inf} for Compound A, or a pharmaceutically acceptable salt thereof, for a patient who is not being administered a strong inducer of CYP3A4.

7. The method of claim 1, wherein the strong inducer of CYP3A4 is chosen from apalutamide, carbamazepine, dexamethasone, enzalutamide, fosphenytoin, lumacaftor, midostaurin, mitotane, pentobarbital, phenobarbital, phenytoin, primidone, rifampicin, rifamycin, rifaximin, rimexolone, and St. John's Wort.

8. The method of claim 7, wherein the strong inducer of CYP3A4 is phenytoin.

9. The method of claim 8, wherein 100 mg phenytoin, measured as the free base, is administered 3 times a day (TID).

10. The method of claim 1, wherein disease or a condition associated with Nav1.6 activity is epilepsy.

11. The method of claim 10, wherein disease or a condition is selected from photosensitive epilepsy, self-induced syncope, intractable epilepsy, Angelman syndrome, benign rolandic epilepsy, CDKL5 disorder, childhood and juvenile absence epilepsy, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures+, Rett syndrome, multiple sclerosis, Alzheimer's disease, autism, ataxia, hypotonia and paroxysmal dyskinesia.

12. The method of claim 10, wherein the disease or condition is epilepsy with focal onset seizures.

13. The method of claim 10, wherein the disease or condition is epilepsy with generalized onset seizures.

14. The method of claim 10, wherein the disease or condition is epilepsy with unknown onset seizures.

15. The method of claim 10, wherein the disease or condition is epileptic syndrome associated with mutations in SCN8A.

16. The method of claim 10, wherein the disease or condition is Dravet syndrome.

17. The method of claim 10, wherein the patient is an adult patient.

18. The method of claim 10, wherein the patient is a pediatric patient.

19. The method of claim 1, further comprising informing the patient or a medical care worker that co-administration of Compound A, or a pharmaceutically acceptable salt thereof, with a strong inducer of CYP3A4 results in no significant changes in the pharmacokinetics of Compound A.

20. The method of claim 1, further comprising informing the patient or a medical care worker that co-administration of Compound A, or a pharmaceutically acceptable salt thereof, with a strong inducer of CYP3A4 results in no significant changes in the pharmacodynamics of Compound A.

21. The method of claim 1, further comprising informing the patient or a medical care worker that dose adjustment is not necessary when Compound A, or a pharmaceutically acceptable salt thereof, is co-administered with a strong inducer of CYP3A4.

22. The method of claim 1, wherein Compound A, or a pharmaceutically acceptable salt thereof, is administered orally.

23. The method of claim 1, wherein Compound A, or a pharmaceutically acceptable salt thereof, is Compound A free base.

24. The method of claim 1, wherein Compound A, or a pharmaceutically acceptable salt thereof, is a pharmaceutically acceptable salt of Compound A.
